(19) United States
(12) Patent Application Publication
(10) Pub. No.: US 2026/0147069 A1
(43) Pub. Date: May 28, 2026

(54) CHARACTERIZING AND MAPPING OF A MAGNETIC FIELD OF A MEDICAL DEVICE

(71) Applicant: STARPAX BIOPHARMA INC., Montréal (CA)

(72) Inventors: Dumitru Loghin, Laval (CA); Maxime Latulippe, Montréal (CA)

(21) Appl. No.: 19/413,950

(22) Filed: Dec. 9, 2025

Related U.S. Application Data
(63) Continuation of application No. PCT/CA2025/051600, filed on Nov. 27, 2025.
(60) Provisional application No. 63/726,079, filed on Nov. 27, 2024.

(51) Int. Cl. G01R 33/02; A61B 34/00; G01R 33/10

(52) U.S. Cl. CPC G01R 33/0206; A61B 34/73; G01R 33/10

(57) ABSTRACT

A method of representing a magnetic field of a magnetic source of a medical device with a computer model of the magnetic field, comprising acquiring, for a plurality of locations, an amplitude value of the magnetic field and corresponding positional coordinates of each of the locations; acquiring a theoretical three-dimensional model of the magnetic field; estimating errors of amplitude between the default values of the theoretical three-dimensional model and the corresponding amplitude values of the plurality of magnetic field measurement entries based on the positional coordinates of the plurality of magnetic field measurement entries; and adjusting default values of the theoretical three-dimensional model based on the estimated errors, resulting in a real-world three-dimensional magnetic field model of the magnetic field of the magnetic source of the medical device from the theoretical three-dimensional model with adjusted amplitude values.

The present application a continuation application of international PCT application No. PCT/CA2025/051600 filed Nov. 27, 2025, designating the United States, that claims priority from U.S. provisional patent application No. 63/726,079 filed on Nov. 27, 2024, the contents of which are incorporated herein by reference.

### TECHNICAL FIELD

The present disclosure relates to the mapping of the magnetic field of a medical device, and more specifically to generating a computer model of a real magnetic field of a magnetic field source of a magnetic-field-based medical device.

### BACKGROUND

Various medical devices can use or generate a magnetic field (MF). The calibration of this magnetic field may be necessary to ensure that the medical device is used properly or that it can produce the required results.

For instance, the following patent documents describe prior art medical applications harnessing a magnetic field: U.S. Pat. No. 9,655,539, U.S. Pat. No. 9,381,063, U.S. Pat. No. 9,220,425, U.S. Pat. No. 8,986,214, U.S. Pat. No. 8,684,010, U.S. Pat. No. 8,457,714, US20130006100, US20120310111, US20120289822, US20120288838, US20110092808, U.S. Pat. No. 7,873,401, US20100305402, US20090275828, US20090248014, US20050096589, etc. In most cases, the accuracy of the magnetic field or of the control of the magnetic field is important.

It is well known by the person skilled in the art that the control and the generation of a magnetic field becomes increasingly more complex and difficult the more MF sources are used to do so. While more MF sources allows for generating a more complex magnetic field, it generally implies that smaller changes in the characteristics (e.g., position, distribution, flux and amplitude) of the magnetic field of each of the MF sources have an even greater impact on the generated magnetic field.

Therefore, the accuracy of the calibration of the magnetic field can rapidly become significantly more complex and critical when the magnetic field used by the medical device is generated by two or more MF sources. In such cases, it can be essential to complete a precise and thorough calibration or characterization of the various MF sources.

On the other hand, various tracking devices are used to track the location of a wide variety of measuring devices that can be a medical device or can be used in combination with one. In the art, the tracking of measuring devices such as a magnetic field, an ultrasound and an image measuring devices can be done in real time. The tracking of the location of these measuring devices can be achieved using various apparatuses or setups, such as cameras, for example.

However, manual mapping of a real-world magnetic field is time-consuming and may be imprecise due to human error. Therefore, a system for mapping a magnetic field in three-dimensional space would be advantageous.

### SUMMARY

The present disclosure relates to methods and systems for mapping a magnetic field generated by one or more magnetic field sources (e.g. magnetic heads) in three-dimensional space.

The present methods and systems harness a probe that measures various magnetic field amplitude readings around a magnetic head. The positions of the probe where the readings are taken may be determined from positional coordinates derived from one or more position sensors. Errors between the measured amplitude readings and default values, at corresponding positional coordinates, of a theoretical three-dimensional model of the magnetic field can be estimated. A real-world three-dimensional model of the magnetic field generated by the magnetic head is then obtained by adjusting the theoretical model based on the estimated errors. In some embodiments, the accuracy of the real-world model is verified and optionally refined.

A broad aspect of the present disclosure is a computing device for representing, with a computer model, a magnetic field generated by a magnetic head of a magnetic-field-based medical device, comprising: 1) an input interface for receiving a plurality of magnetic field measurement entries of the magnetic field; 2) a processor; and 3) memory storing program code that, when executed by the processor, causes the processor to: A) receive a theoretical three-dimensional model of the magnetic field generated by the magnetic head; B) receive, via the input interface, the plurality of magnetic field measurement entries, wherein each magnetic field measurement entry of the plurality of magnetic field measurement entries comprises an amplitude value of the magnetic field and a corresponding positional coordinate; C) estimate an error of amplitude between default amplitude values of the theoretical three-dimensional model and the corresponding amplitude values of the plurality of magnetic field measurement entries based on the positional coordinates of the plurality of magnetic field measurement entries; and D) adjust default values of the theoretical three-dimensional model based on the estimated errors, resulting in a real-world three-dimensional magnetic field model from the theoretical three-dimensional model with adjusted amplitude values, whereby the real-world three-dimensional magnetic field model comprises a constellation of adjusted amplitude values in a space representative of the magnetic field generated by the magnetic head.

In some embodiments, the estimating may include calculating a difference between the default values of the magnetic field of the theoretical model and the amplitude values of the magnetic field of the plurality of magnetic field measurement entries through an interpolation method.

In some embodiments, the adjusting may be based on a Kriging interpolation method.

In some embodiments, the computing device may include the medical device.

In some embodiments, the input data may further include a value of orientation of the magnetic field, and wherein the estimating the errors may further use the value of orientation of each of the correlated the input data.

In some embodiments, the program code, when executed by the processor, may further cause the processor to verify an accuracy of the real-world three-dimensional magnetic field model of the magnetic field.

In some embodiments, the verifying an accuracy of the real-world three-dimensional magnetic field model of the magnetic field may include: A) receiving at least one additional magnetic field measurement entry comprising an amplitude value of the magnetic field and a corresponding positional coordinate; B) estimating at least one additional error of amplitude between the default amplitude values of the real-world three-dimensional magnetic field model and at least one corresponding additional real-world amplitude value, wherein the at least one corresponding additional real-world amplitude value is determined from at least one additional magnetic field measurement entry including a corresponding positional coordinate; and C) determining if the at least one additional error satisfies a verification threshold value to verify the accuracy of the real-world three-dimensional magnetic field model of the magnetic field.

In some embodiments, the program code, when executed by the processor, may further cause the processor to refine the real-world three-dimensional magnetic field model of the magnetic field.

In some embodiments, the program code, when executed by the processor, may further cause the processor to refine the real-world three-dimensional magnetic field model of the magnetic field when the real-world three-dimensional magnetic field model of the magnetic field does not satisfy the verification of the accuracy of the real-world three-dimensional magnetic field model of the magnetic field.

In some embodiments, the refining of the real-world three-dimensional magnetic field model of the magnetic field may include: A) receiving the at least one additional magnetic field measurement entry comprising the amplitude value of the magnetic field and the corresponding positional coordinate; B) estimating the at least one additional error of amplitude between the amplitude values of the real-world three-dimensional magnetic field model and the at least one corresponding real-world amplitude value of the at least one additional magnetic field measurement entry; and C) adjusting the amplitude values of the real-world three-dimensional magnetic field model based on the at least one estimated additional error resulting in a refined real world three-dimensional magnetic field model.

In some embodiments, the verifying may be repeated after each of the refining to verify an accuracy of the resulting refined real-world three-dimensional magnetic field model and wherein the refining of the real-world three-dimensional magnetic field model of the magnetic field is repeated until the refined real-world three-dimensional magnetic field model satisfies the verification threshold.

In some embodiments, the data may be acquired with: 1) a magnetic field measuring device able to measure an amplitude of a magnetic field; and 2) a tracking device for identifying a location of the magnetic field measuring device; 3) wherein the magnetic field measuring device is used for determining the amplitude value of the magnetic field, and wherein the tracking device may be used to measure the location of the magnetic field measuring device to determine the corresponding positional coordinates.

In some embodiments, the magnetic field measuring device may further measure the value of orientation of the magnetic field.

In some embodiments, the magnetic field measuring device may be a gaussmeter.

In some embodiments, the magnetic field measuring device may be coupled to at least one location marker that is trackable by the tracking device and wherein the tracking device determines a location of the at least one location marker to determine the location of the magnetic field measuring device.

In some embodiments, the at least one location marker may be mounted on a frame coupled to the magnetic field measuring device, wherein the at least one location marker may be positioned and the frame may be shaped so as to reduce uncertainties of the location.

In some embodiments, the magnetic field measuring device may be coupled to the medical device for the measuring the amplitude value of the magnetic field.

In some embodiments, the magnetic field measuring device may be coupled to the medical device by way of a position stage, wherein the position stage may be used to change the location of the magnetic field measuring device relative to the magnetic field of the magnetic head of the medical device.

In some embodiments, the magnetic field measuring device may be coupled to a patient table of the medical device and wherein the table may be moved to change the location of the magnetic field measuring device relative to the magnetic field of the magnetic head of the medical device.

In some embodiments, the location of the magnetic field measuring device may be comprised within a working region of the medical device.

In some embodiments, the input interface may be connectable to another computing device or another memory storing the data and providing the data to the processor.

In some embodiments, the at least one location marker may be a reflective marker, and wherein the tracking device may be a stereo camera tracking the location of one or more of the at least one location marker to determine the location of the magnetic field measuring device.

In some embodiments, the at least one location marker may include at least three location markers, wherein the corresponding positional coordinates may be determined by triangulation using the locations of the at least three location markers determined by the tracking device.

In some embodiments, the magnetic head may be a first magnetic head, and the representation of the magnetic field of the magnetic head of the medical device may be used with a representation of a magnetic field of at least one other magnetic head of the medical device to generate a computer model of a combined magnetic field comprising the magnetic field of the first magnetic head and of at least the magnetic field of the at least one other magnetic head.

In some embodiments, each of the magnetic heads and the at least one other magnetic head may include at least one electromagnet.

In some embodiments, the magnetic-field-based medical device may include three pairs of magnetic heads comprising the first magnetic head and the at least one other magnetic head may include five magnetic heads.

In some embodiments, the three pairs of magnetic heads may be orthogonal with respect to each of the other two pairs of the three pairs.

In some embodiments, the medical device may be a system for steering magnetotactic entities in a subject.

Another broad aspect is a method of representing a magnetic field of a magnetic source of a medical device with a computer model of the magnetic field, comprising: 1) acquiring, for a plurality of locations, an amplitude value of the magnetic field and corresponding positional coordinates of each of the locations; 2) acquiring a theoretical three-dimensional model of the magnetic field; 3) estimating errors of amplitude between the default values of the theoretical three-dimensional model and the corresponding amplitude values of the plurality of magnetic field measurement entries based on the positional coordinates of the plurality of magnetic field measurement entries; and 4) adjust default values of the theoretical three-dimensional model based on the estimated errors, resulting in a real-world three-dimensional magnetic field model of the magnetic field of the magnetic source of the medical device from the theoretical three-dimensional model with adjusted amplitude values.

In some embodiments, the acquiring the amplitude value of the magnetic field and the corresponding positional coordinates may include: I) providing a magnetic field measuring device that can measure an amplitude of the magnetic field; II) providing a tracking device that can determine a location of the magnetic field measuring device; III) measuring, for each of the plurality of locations, the amplitude value of the magnetic field using the magnetic field measuring device; and IV) acquiring, for each of the plurality of locations, the corresponding positional coordinates of each of the measured amplitude values by determining, using the tracking device, a location of the magnetic field measuring device when completing each of the measuring of the amplitude values, and wherein the proposed method further comprises: 1) providing the medical device; and 2) generating a magnetic field with a magnetic field source of the medical device.

In some embodiments, the proposed method may include at least one of: verifying an accuracy of the real-world three-dimensional magnetic field model of the magnetic field; and refining the real-world three-dimensional magnetic field model.

In some embodiments, the refining may be performed if the real-world three-dimensional magnetic field model does not satisfy the verifying the accuracy.

In some embodiments, the verifying may include: A) providing at least one additional amplitude value of the magnetic field for at least one additional location and additional corresponding positional coordinates; B) for each on the at least one additional amplitude value, estimating an additional error of amplitude between the at least one additional amplitude value and an amplitude of the real-world three-dimensional magnetic field model based on positional coordinates of the real-world three-dimensional magnetic field model; and C) determining if each of the estimated additional error of amplitude satisfies a verification threshold value to verify an accuracy of the real-world three-dimensional magnetic field model.

In some embodiments, the refining may include: A) providing at least one additional amplitude value of the magnetic field for at least one additional location and additional corresponding positional coordinates; B) for each on the at least one additional amplitude value, estimating an additional error of amplitude between the at least one additional amplitude value and an amplitude of the real-world three-dimensional magnetic field model based on positional coordinates of the real-world three-dimensional magnetic field model; and C) adjusting the amplitude of the real-world three-dimensional magnetic field model based on the estimated additional error of amplitude.

In some embodiments, the refining may be repeated, with at least one further additional amplitude value of the magnetic field for at least one further additional location and further additional corresponding positional coordinates, until the real-world three-dimensional magnetic field model satisfies the verifying the accuracy.

In some embodiments, the proposed method may include generating a complete three-dimensional magnetic field model of a complete magnetic field of the medical device generated by a plurality of the magnetic field source by combining the real-world three-dimensional magnetic field model of each of the plurality of the magnetic field source.

In some embodiments, the proposed method may include sending the control parameters to controllers of the medical device, each controlling one magnetic field source of the plurality of magnetic field sources.

Another broad aspect is non-transitory computer-readable medium having stored thereon program instructions representing a magnetic field of a magnetic source of a medical device with a computer model of the magnetic field, the program instructions executable by a processing unit for: acquiring, for a plurality of locations, an amplitude value of the magnetic field and corresponding positional coordinates of each of the locations; acquiring a theoretical three-dimensional model of the magnetic field; estimating errors of amplitude between the default values of the theoretical three-dimensional model and the corresponding amplitude values of the plurality of magnetic field measurement entries based on the positional coordinates of the plurality of magnetic field measurement entries; and adjusting default values of the theoretical three-dimensional model based on the estimated errors, resulting in a real-world three-dimensional magnetic field model of the magnetic field of the magnetic source of the medical device from the theoretical three-dimensional model with adjusted amplitude values.

In some embodiments, the program instructions may be further executable by a processing unit for providing the medical device; and generating a magnetic field with a magnetic field source of the medical device; wherein the acquiring the amplitude value of the magnetic field and the corresponding positional coordinates may include providing a magnetic field measuring device able to measure an amplitude of the magnetic field; providing a tracking device able to determine a location of the magnetic field measuring device; measuring, for each of the plurality of locations, the amplitude value of the magnetic field using the magnetic field measuring device; and acquiring, for each of the plurality of locations, the corresponding positional coordinates of each of the measured amplitude values by determining, using the tracking device, a location of the magnetic field measuring device when completing each of the measuring of the amplitude values.

In some embodiments, the program instructions may be further executable by a processing unit for verifying an accuracy of the real-world three-dimensional magnetic field model of the magnetic field; and refining the real-world three-dimensional magnetic field model of the magnetic field.

In some embodiments, the refining may be performed if the real-world three-dimensional magnetic field model does not satisfy the verifying the accuracy.

In some embodiments, the verifying may include receiving at least one additional amplitude value of the magnetic field for at least one additional location and additional corresponding positional coordinates; for each of the at least one additional amplitude value, estimating an additional error of amplitude between the at least one additional amplitude value and an amplitude of the real-world three-dimensional magnetic field model based on positional coordinates of the real-world three-dimensional magnetic field model; and determining if each of the estimated additional error of amplitude satisfies a verification threshold value to verify an accuracy of the real-world three-dimensional magnetic field model.

In some embodiments, the refining may include providing at least one additional amplitude value of the magnetic field for at least one additional location and additional corresponding positional coordinates; for each on the at least one additional amplitude value, estimating an additional error of amplitude between the at least one additional amplitude value and an amplitude of the real-world three-dimensional magnetic field model based on positional coordinates of the real-world three-dimensional magnetic field model; and adjusting the amplitude of the real-world three-dimensional magnetic field model based on the estimated additional error of amplitude.

In some embodiments, the refining may be repeated, with at least one further additional amplitude value of the magnetic field for at least one further additional location and further additional corresponding positional coordinates, until the real-world three-dimensional magnetic field model satisfies the verifying the accuracy.

In some embodiments, the program instructions may be further executable by a processing unit for generating a complete three-dimensional magnetic field model of a complete magnetic field of the medical device generated by a plurality of the magnetic field sources by combining the real-world three-dimensional magnetic field model of each of the plurality of the magnetic field sources.

In some embodiments, the program instructions may be further executable by a processing unit for sending the control parameters to controllers of the medical device, each controlling one magnetic field source of the plurality of magnetic field sources.

Another broad aspect is a medical system comprising magnetic heads of which the magnetic field of each of the magnetic heads has been defined by performing the method described herein.

### BRIEF DESCRIPTION OF THE DRAWINGS

The invention will be better understood by way of the following detailed description of embodiments of the invention with reference to the appended drawings, in which:

FIG. 1A is a block diagram of an exemplary apparatus of the proposed computing device for representing a magnetic field of a medical device with a computer model;

FIG. 1B is a block diagram of another exemplary apparatus of the proposed computing device for representing, with a computer model, a magnetic field of at least one magnetic head of a medical device, where magnetic field measurement entries are directly provided from a magnetic field measuring device and a location tracking device;

FIG. 1C is a block diagram of another exemplary apparatus of the proposed computing device for representing, with a computer model, a magnetic field of at least one magnetic head of a medical device, where magnetic field measurement entries are provided by a data processing unit;

FIG. 2A is a flowchart diagram of an exemplary method of generating a computer model of a magnetic field of a medical device;

FIG. 2B is a flowchart diagram of another exemplary method of generating, verifying and/or refining a computer model of a magnetic field of a medical device;

FIG. 3 is a flowchart diagram of an exemplary method of generating a complete magnetic field with a plurality of MF sources of a magnetic-field-based medical device;

FIG. 4A is a schematic representation illustrating an example of a step of correlating measurement entries of the real magnetic field with points of the theoretical model of the magnetic field;

FIG. 4B is a schematic representation illustrating an example of a step of interpolating theoretical model of the magnetic field with the correlated measurements entries;

FIG. 5 is a schematic representation illustrating a perspective view of an example of a supporting device for positioning the magnetic field measuring device and trackable by the location tracking device;

FIG. 6A is a block diagram of an exemplary magnetic-field-based medical device;

FIG. 6B is a block diagram of an exemplary magnetic-field-based medical device comprising an exemplary embodiment of the proposed computing device for representing a magnetic field;

FIG. 7A is a drawing of a front view of an exemplary magnetic-field-based medical device for steering magnetotactic entities;

FIG. 7B is a drawing of a perspective view of the exemplary magnetic-field-based medical device of FIG. 7A;

FIG. 8A is a schematic representation illustrating a perspective view of the contraption of FIG. 5 positioned in the working region and coupled to the patient table of the medical device of FIG. 7A;

FIG. 8B is a schematic representation illustrating a front view of the supporting device of FIG. 5 positioned in the working region of the medical device of FIG. 7A; and

FIG. 8C is a schematic representation illustrating a side view of the supporting device of FIG. 5 positioned in the working region of the medical device of FIG. 7A.

### DETAILED DESCRIPTION

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

From the foregoing it will be appreciated that, although specific embodiments have been described herein for purposes of illustration, various modifications may be made without deviating from the spirit and scope of the teachings. Accordingly, the claims are not limited by the disclosed embodiments.

The embodiments are in such detail as to clearly communicate the disclosure without limiting the anticipated variations of the possible embodiments and may encompass all modifications, equivalents, combinations and alternatives falling within the spirit and scope of the present disclosure. It will be appreciated by those skilled in the art that well-known methods, procedures, physical processes and magnetic heads may not have been described in detail in the following so as not to obscure the specific details of the disclosed invention.

It will be appreciated that it is generally complex to accurately predict the real-world characteristics of a magnetic field from theoretical knowledge. In fact, theoretical representations of magnetic fields are generally resulting from numerous approximations and assumptions. Therefore, in order to prevent significant deviations from the theoretical models of a magnetic field, the characterization (mapping) of the real-world magnetic fields produced by the various MF sources of the medical device may be very important or, in some cases, critical.

Magnetic fields can be varied and may become increasingly more complex when interacting with additional magnetic fields. A person skilled in the art will appreciate that relying on the computer models mapping the real-world magnetic field (actually generated by the various MF sources), with all their respective/varied/multiple imperfections, can significantly improve the accuracy of the complete computer model and therefore increase the accuracy of the real-world complete magnetic field. This can result in a significantly more accurate medical device and treatment.

The imperfections of the magnetic field here relate to any characteristic (distribution, shape, amplitude, asymmetry, jittering/fluctuations, etc.) of the real-world magnetic field that may be diverging from an ideal/theoretical magnetic field that would be generated by an ideal/perfect MF source. Such imperfections of the magnetic field can result from any imperfection of the MF source (e.g., any imperfection, loose production tolerances, low quality control, degradation/aging and/or environmental condition that may be associated with or can affect a material, component or assembly of the MF source) or anything that may interact with the magnetic field (e.g., any other magnetic field, any electric field, any magnetic material or any diamagnetic material).

To ensure that an accurate magnetic field is generated by the medical device, the proposed method and apparatus can be used to accurately map with a computer model the real-world magnetic field generated by a MF source through measuring the magnetic field's characteristics at various positions in 3D space while tracking these positions. A complete 3D computer model (comprising multiple MFs) may be generated that accurately represents (maps) the real (imperfect) magnetic field produced by the medical device. This can enable the accurate control of the complete magnetic field when using/operating the medical device. The control of the MF sources can be done by considering the actual/real impact on the individual and/or the complete magnetic field generated by the medical device.

It will be appreciated that a position or a location, which may be used interchangeably in the present document when the context allows it, are defined herein as a particular/precise point in space (two-dimensional or three-dimensional space) that can be characterized by two or three coordinates of a chosen coordinate system.

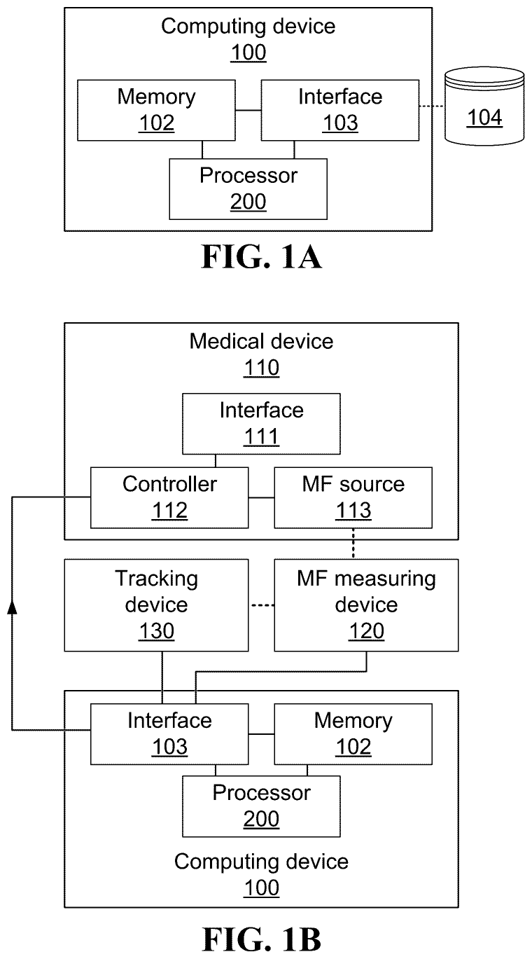

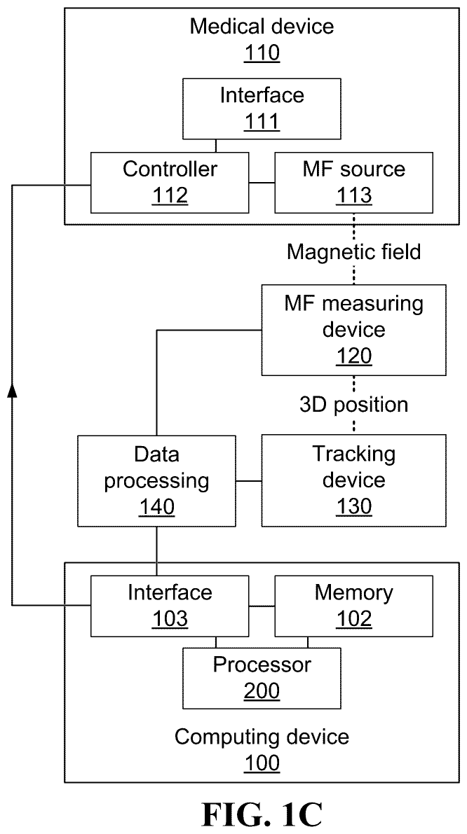

Now referring to FIG. 1A that shows a block diagram of an exemplary embodiment of the proposed computing device 100 for representing/mapping a magnetic field (MF) of a medical device with a computer model. The proposed computing device 100 can comprise at least one processor 200, a memory 102 and an input interface 103 able to at least receive measurement entries. These elements/modules can all be directly and/or indirectly connected and/or coupled with one another. Two or more of these elements can be interconnected via any suitable means known in the art (integrated with each other, cables, internet, ethernet, Wi-Fi, Bluetooth®, etc.). Two or more of these elements can be integrated within a same housing or module.

The processor 200 of the computing device 100 can be any suitable general-purpose programmable processing unit known in the art (microprocessor, DSP, FPGA, etc.). The processor can be configured to receive and perform instructions from the memory 102. In this example, the processor is shown as being unitary, but the processor may also be multicore, or distributed (e.g., a multi-processor).

The memory 102 of the computing device 100 can be used to save any relevant data (e.g., any data acquired or generated by the processor) and/or to store at least one program code (program instructions and commands) to properly operate the computing device 100 and/or any other type of required information. In some embodiments, the memory can be any suitable type of transitory and/or non-transitory memory known in the art, which may be at least one of: random-access memory (RAM), read-only memory (ROM), solid-state drive (SSD), hard disk drive (HDD), a combination thereof, etc. In some embodiments, the memory 102 can comprise a plurality of memory layers. The memory can optionally store a theoretical computer model of an ideal/theoretical/expected magnetic field of one or more magnetic heads of a medical device.

The interface 103 of the proposed computing device 100 can be configured to ensure that it can at least receive—from at least one external apparatus—measurement entries, which may at least include magnetic field amplitude values and corresponding positional coordinates. The interface can optionally receive a theoretical computer model of a theoretical magnetic field of one or more magnetic heads of a medical device. The input interface may be connectable/compatible with a wide variety of apparatuses (illustrated in FIG. 1A as a block representing any combination of external, optional and suitable apparatus 104 configured to exchange data with the interface 103), which can be, but is not limited to, one or more of: a database, a memory module, a medical device, a controller of a medical device, a computer, a data processing unit, a magnetic field measuring device or a location identifying/detecting device. It will be appreciated that the interface can communicate and be connected to such apparatuses using any suitable means known in the art (cables, internet, ethernet, Wi-Fi, Bluetooth®, USB, etc.). In some embodiments, the interface may comprise an input and/or an output function so as to enable both input and output of information/data. For example, the interface can be used to send input data to the memory 102 and/or the processor 200. In some embodiments, the interface can be used to output data from the memory 102 and/or from the processor 200, such as a completed computer model of a magnetic field generated from the processor, for example.

As illustrated in FIGS. 1B and 1C, in some embodiments, the proposed computing device 100 can be connected (e.g., through its interface 103) to various other apparatuses. In the embodiment of FIG. 1B, the computing device 100 can be directly connected to various measuring devices (e.g., a magnetic field measuring device 120 and/or a tracking device 130), the medical device 110, which may comprise at least one MF source.

The magnetic field measuring device 120 or probe may be any device or setup that can allow for the measurement of one or more of the characteristics of the magnetic field generated by a MF source 113 of the medical device 110. The measured characteristics can include, but are not limited to, a local amplitude, an averaged amplitude, a local direction/orientation, a local flux, a force, any value of variation over time, or a combination thereof.

In some embodiments, the magnetic field measuring device 120 can be, but is not limited to, a magnetometer that can be configured to measure at least one of a direction, strength, or relative change of a magnetic field at a particular location. The magnetometer can be at least one gaussmeter or at least one teslameter.

The tracking device 130 may be any device or setup that can allow to track/identify/determine the location—preferably in 3D space and with satisfying accuracy—of the magnetic field measuring device 120 in order to associate to each of the tracked positions, a corresponding measurement/acquisition/reading (e.g., a value of amplitude) of the magnetic field. In some embodiments, the tracking device 130 can comprise a mechanical and/or a remote tracking device.

A mechanical tracking device may be, but is not limited to, a robotic device (e.g. articulated or CNC robotic arm, CMM arm, collaborative robot, etc.) to which the magnetic field measuring device is directly or indirectly coupled that allows an operator to position the magnetic field measuring device at various determined locations. In some cases the mechanical tracking device can be calibrated using the position of the MF source in order to ensure that the measured/tracked position/location can be accurately related or compared to the position of the MF source. In an exemplary embodiment where the mechanical tracking device is a robotic arm, the calibration can comprise associating the position of the MF source (e.g., a tip of a magnetic head) to the origin (x=0, y=0 and z=0) of the reference coordinates (reference axes) of the tracking measurements and system.

A remote tracking device may rely on electromagnetic waves and signals (laser(s), radio waves, Wi-Fi, Bluetooth®, infrared, visual light, camera, photosensors, etc.) that can rely on triangulation to determine the location in 3D space. The triangulation may be achieved by using at least three fixed receivers/detectors receiving information—in communication with—at least one location marker coupled to the object to be tracked (moving object). Alternatively, the triangulation can be achieved by using at least one fixed receiver receiving information—in communication with—at least three location markers in a fixed location relative to the object to be tracked (moving object). For example, a visual tracking device can rely on a visual receiver such as one or more cameras (e.g. photosensor, grey scale camera multispectral camera, stereo camera, RGB camera, infrared camera, etc.) to capture and identify the location of at least three location markers (reflective spheres, color markers, etc.) that may be affixed in an asymmetric arrangement to the magnetic field measuring device in order to determine and track its 3D location by triangulation of the at least three location markers.

In an embodiment, the location tracking device(s) and/or the location marker(s) may be part of or coupled to a frame configured to hold/support or to be coupled with the magnetic field measuring device 120. The location markers can be positioned at a fixed position relative to the magnetic field measuring device 120. It will be appreciated by someone skilled in the art that the location markers can be arranged in an asymmetric layout in order to facilitate recognition of the position of the magnetic field measurement device and/or of the supporting frame, especially when detected at different orientations (from various perspectives). For instance, correspondences between image objects of the location markers in image space, as captured by the camera, is determined. Then, relative positions of the image objects of the location markers in image space is compared to a model of the location markers (with respect to one another) to determine orientation and/or translation.

The frame can be designed and configured to reduce possible uncertainties on location measurements that may arise when measuring the various positions of the location marker(s). In some embodiments, uncertainties (e.g., on the 3D coordinates measurements—on the x/y/z axes) can be reduced by selecting an appropriate shape for the frame coupled with the magnetic field measuring device. In some cases, the frame can have dimensions that may be significantly larger than the dimensions of the magnetic field measuring device, in order to take advantage of a possible reduction of the relative uncertainty of measurements (e.g., similar absolute error/uncertainty for a distance measurement over a measured distance selected to be larger) of the location of a location marker, which may result in an improved precision and/or orientation (i.e., reduced uncertainty) for the measurements/calculations of the location of the magnetic field measuring device.

This frame, supporting frame, can be a position stage (e.g., manual or automated linear millimeters position stage.) that can be used to control the movement/displacement of the frame and/or of the magnetic field measuring device. A supporting frame that can be used as a position stage may have various advantages and may give the operator (user of the proposed apparatus and method) the capacity to incrementally modify the location of the magnetic field measuring device, to adjust its location in 1D, 2D or 3D, to prevent any undesired location variations (e.g., to prevent wobbling), to maintain it at a given location for an extended period of time if required, and to give him overall better control over the location and orientation of the magnetic field measuring device.

It will be appreciated that, in some embodiments, both measurements (of the location and of the magnetic field) can be completed simultaneously. A trigger may be connected to both the tracking device and the magnetic field measuring device to trigger the measurements and, optionally, to ensure simultaneous acquisition of both of the measurements.

The medical device 110 can comprise an interface 111, at least one MF source 113 (e.g., magnetic head), and at least one controller 112. The MF source 113 can be any apparatus enabling generating a magnetic field, which can comprise one or more magnets or electromagnets. The controller 112, which can comprise a switch and/or a current modulator (e.g., including current amplifiers; bipolar current amplifiers, etc.), may be used to control the flow of the electric current to the at least one MF source, in order to cause the generation, or modifying the generation of the corresponding magnetic field. The interface 111 of the medical device can include an input, an output, and/or a user/operator interface. The interface can be configured to connect to various devices such as a computer, memory (database, USB key, memory card, etc.) and to some of the other components of the medical device (which can be, but are not limited to, the MF source and/or the controller). The interface can be operable by a user/operator in order to use and control the medical device.

In some embodiments, as illustrated in the example of FIG. 1C, the measuring devices (location measuring/tracking device and/or magnetic field measuring device) may be connected to a data processing unit/module 140 that may be connectable to the various measuring devices (e.g., the magnetic field measuring device 120 and the tracking device 130) so that the raw measurements (data output of the measuring devices) may be processed before being sent to the computing device 100.

The processing of the raw measurements can include, but is not limited to, filtering the data (e.g., averaging, band pass filtering, low pass filtering, high pass filtering, noise filtering), associating the data of the various measuring devices with one another (for example, arranging the measurement entries so that each data of the measured characteristic of the magnetic field is paired with the data of the tracked location of the corresponding measuring), classifying/ordering the data, or a combination thereof. The data processing module 140 can comprise any apparatus allowing for the proper management of the data. In some embodiments, the data processing module 140 can be an off-the-shelf computing device, a programmable computing device, and may comprise an interface, a memory and a processor. In some embodiments, the data processing module can be configured to allow (e.g., with a controller) to control the various measuring devices and may allow for simultaneous acquisition with these measuring devices.

In one embodiment, the various measuring devices and optionally the data processing unit may all be part of a single measuring apparatus that would be used to provide the measurement entries to the computing device 100.

In one embodiment, the computing device 100 can include the various measuring devices 120, the tracking device 130 and/or optionally the data processing unit 140.

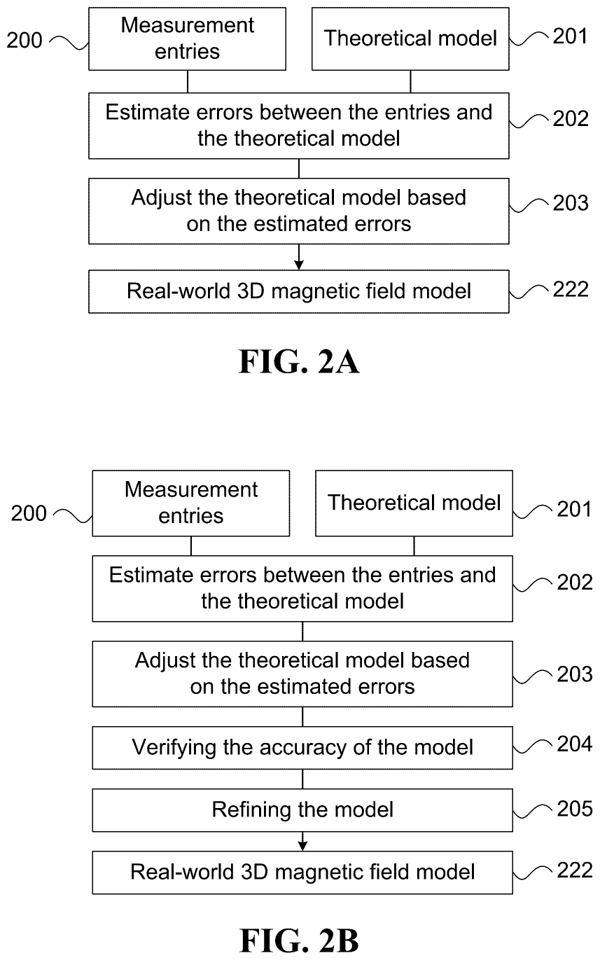

Reference is now made to FIG. 2A that presents steps that may be completed to perform an exemplary method of representing/mapping a magnetic field of the MF source 113 of a medical device 110 with a computer model (e.g., generating a real-world three-dimensional magnetic field model at step 222). The method can include a step 200 of acquiring measurement entries that can comprise acquiring, for a plurality of locations, information (e.g., a value of amplitude) about a magnetic field and corresponding coordinates; a step 201 of acquiring a theoretical model of the magnetic field, which can be a theoretical 3D computer model of an ideal magnetic field; a step 202 of estimating errors of magnetic field characteristic (e.g., amplitude) values between the default values of the acquired theoretical model and a corresponding (e.g., at a corresponding coordinate) real-word characteristic (e.g., amplitude) value based on the measured characteristics of the measurement entries; and a step 203 of adjusting the theoretical model based on the estimated errors to produce a real-world three-dimensional magnetic field model 222.

The measurement entries can comprise various information/data relating to the various characteristics of the measured magnetic field and/or relating to the state of the magnetic field measuring device when a measurement/acquisition is completed. In some embodiments, the measurement entries comprise at least an amplitude value of said magnetic field and a corresponding positional coordinate.

The real-word characteristic values may include the measured characteristic values of the measurement entries, intermediate characteristic values (i.e., values for the intermediate coordinates-space/coordinates between the coordinates—of the measurement entries), or a combination thereof. In some embodiments, the intermediate characteristic values (intermediate values) may be acquired (e.g., taken from a provided dataset or a data server) or can be determined (e.g., calculated or estimated) based on the various measured characteristics of the measurement entries. For example, the intermediate values may be determined by selecting and using a function (e.g., linear, polynomial or parabolic) to approximate the values of the real-world characteristic of the intermediate coordinates (between the coordinates of the measurement entries) from the measured values of the measurement entries (e.g., by fitting the selected function with at least some of the measured values). In some embodiments, these intermediate values can be used to complete a populating sub-step and may be used to increase/adjust the resolution of the measurement entries to match the resolution of the theoretical model (e.g., to calculate and/or populate the intermediate values corresponding to the coordinates of the defaults values of the theoretical model).

The estimating of step 202 can comprise determining, approximating, calculating and/or selecting the value of the estimated error, which may be a gap, a difference, change, disparity, variation, etc. between the default values of the characteristics of the theoretical model and the values of the real-word characteristics. In some embodiments, step 202 may comprise a sub-step of populating a three-dimensional environment with the measured characteristics of the measurement entries at the corresponding positional coordinates. In some embodiments, three-dimensional environment may be populated alternatively or additionally with intermediate characteristic values. In some embodiments where the theoretical model is a three-dimensional virtual computer model, the virtual space of the theoretical model may be directly populated with the real-world characteristic values.

It will be appreciated that this optional populating step or any of the steps of the proposed method does not have to be completed in a visual three-dimensional environment and may be done for a non-visual three-dimensional environment such as, for example, a dataset comprising positional information (e.g., coordinates) that may take the form of vectors or matrices.

In some embodiments, step 202 can comprise estimating the errors between the values of real-world characteristics (e.g., the calculated/estimated intermediate values and/or the measured values of the measurement entries), which may include some of the measured values, and the default values of the theoretical model at the corresponding coordinates. This can result in a dataset comprising the error information and their associated coordinates (e.g., a 3D model populated with the error values).

In some embodiments, step 202 may include comparing the measured values with values defined in the theoretical model. These measured values may be interpolated into the theoretical model to determine an error between the measured values and the corresponding values of the theoretical model. The theoretical model is then refined in accordance with the determined errors to determine at any point in the theoretical model the error at that point, permitting a granular refinement in the values of the theoretical model at these points (e.g. through an interpolation of the calculated errors), resulting in an improved theoretical model of the magnetic field.

It will be appreciated that the points of the theoretical model can correspond to a preset resolution, a chosen resolution and/or a vortex of a voxel of the 3D model.

In some embodiments, step 203 can comprise adjusting (e.g., interpolating) the theoretical model to be representative of the magnetic field by considering the error values estimated from the amplitude of the magnetic field correlated with the points of the theoretical model. In some embodiments, the interpolation can be done by considering the amplitude value of each of the correlated measurement entries to adjust the corresponding correlated point of the theoretical model. In some embodiments, step 203 can comprise adjusting the theoretical model based on the estimated errors determined from the intermediate values and the default values of the theoretical model.

In some embodiments, the adjusting can be performed by correcting the various default values of the theoretical model by adding/subtracting the value of the corresponding error. In one embodiment, where the theoretical model is based at least in part on theoretical equations, the adjusting may be performed by modifying (e.g., iteratively and/or in a feedback loop to reduce the values of the estimated errors) at least one equation parameter of the theoretical equation so as to ensure a better/improved/accurate fit between the values of measurement entries and the adjusted model (“real-world” model).

The adjusting (e.g., interpolation) of the theoretical model of step 203 can result in a real-world three-dimensional magnetic field model 222 that is a representation (e.g., a 3D mapping) of the magnetic field generated by the MF source 113. In one embodiment, the real-world three-dimensional magnetic field model 222 can comprise a constellation of adjusted amplitude values, its location in the constellation defined in accordance with its positional coordinate (default values adjusted based on the corresponding estimated error) in a space representative of the magnetic field generated by the magnetic head.

The refinement can be any suitable interpolation, regression (e.g. statistic and/or machine learning based regression), correlation and/or other method (e.g., finite strain theory, strain tensors, transformation matrix such as non-rigid transformations and/or identification/recognition of the magnetic field lines to define a deformation matrix to adjust the model) that may be deemed suitable by a person skilled in the art. In an embodiment, interpolation can be closely related to regression analysis and can, for example, predict the value of a function of the theoretical computer model at a given point (e.g., between the points correlated with a measurement entry) by computing a weighted average of the known values of the function in the neighborhood of the point (e.g., magnetic field characteristic of the correlated points of the measurement entries).

In a preferred embodiment, the interpolation can rely on known and proven methods, such as the Kriging interpolation method (also known as the Gaussian process regression or the Wiener-Kolmogorov prediction) or its derivative or a combination with other interpolation methods and concepts.

It will be appreciated that the proposed method may not be limited to these steps. The method can further comprise additional following steps, preceding steps (e.g., data processing steps and/or measuring steps) and/or intermediate steps (e.g., validation steps and/or filtering steps).

Now referring to FIG. 2B that illustrates another exemplary embodiment of a method comprising some of the possible additional following steps that can be but are not limited to: a step 204 of verifying the accuracy of the model, and an optional following step 205 of refining the real-world three-dimensional magnetic field model 222.

In some embodiments, step 204 can comprise acquiring additional measurement entries and determining if an additional error value (e.g., difference in amplitude between the amplitude value of an additional magnetic field measurement entry and a corresponding amplitude of the real-world three-dimensional magnetic field model 222) satisfies a verification threshold value in order to verify and/or assess the accuracy of the generated real-world three-dimensional magnetic field model.

The verification threshold can be any suitable criteria known in the art that can enable assessing the quality of a three-dimensional computing model. In some embodiment, the verification threshold can be a maximal difference of amplitude values, at or near positional coordinates, between the computer model being verified and an additional amplitude value of an additional measurement entry. In some embodiments, the verification threshold can be a threshold of a coefficient of determination, such as, for example, a mean square error (MSE), a root mean square error (RMSE), a mean absolute error (MAE), etc. The coefficient of determination may be determined/calculated by considering determined differences of amplitude (e.g., by calculating a coefficient of determination a histogram of the calculated differences). The coefficient of determination may be determined/calculated by considering at least a part/section of a magnetic field line of the computing model being verified and some additional measurements (e.g., additional amplitude value).

In some embodiments, step 205 can comprise producing a “refined” computer model (e.g., a “refined” real-world three-dimensional magnetic field model 222) with the initially correlated measurement entries and with at least one of additional correlated magnetic field measurement entry. It will be appreciated that this refining step may be conditional to the verification/assessment of the quality of the generated real-world three-dimensional magnetic field model (e.g., may be performed when the difference in amplitude does not satisfy the verification threshold value).

In some embodiments, the refining step 205 can comprise using an additional amplitude value and corresponding positional coordinates of at least one additional location that is correlated with a corresponding point of the computer model (e.g., correlated with the point of the computer model nearest (e.g., having the smallest difference between location) to the positional coordinates of the additional location(s)).

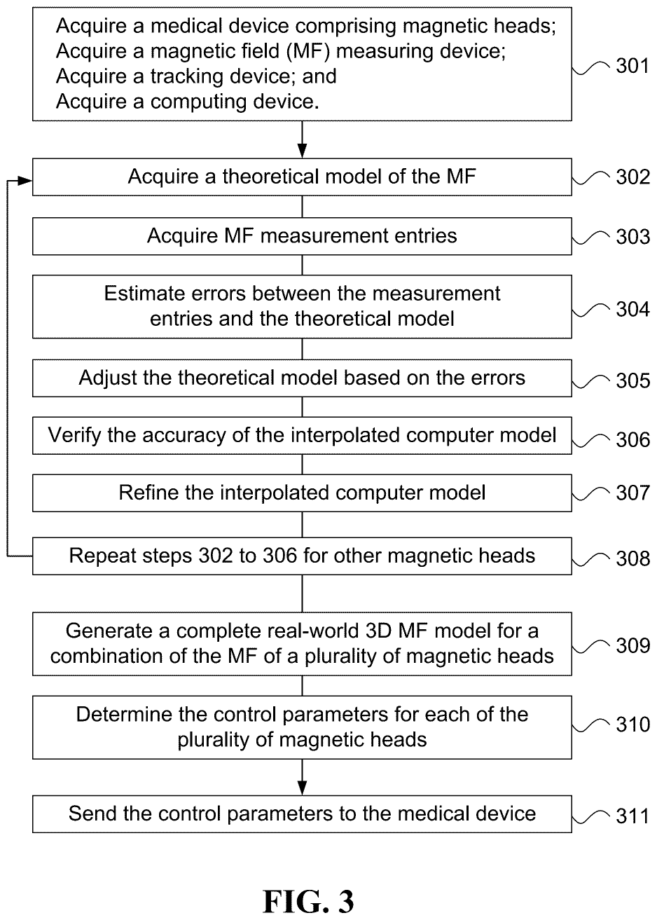

FIG. 3 shows the various steps of an exemplary method that can be performed in a broader context that may consider a medical device 110 having a plurality of MF sources 113, which may each simultaneously produce its own magnetic field that interacts with all of the other magnetic fields to produce a complex (“complete”) magnetic field. The steps can then be but are not limited to: a step 301 of acquiring the necessary tools and devices, which can include, but is not limited to, acquiring at least one of the medical devices comprising the plurality of MF sources (e.g., at least two magnetic heads), a magnetic field measuring device (e.g., a magnetometer), a tracking device (e.g., a camera for tracking at least three reflective location markers coupled to the magnetic field measuring device) and a computing device for performing the proposed method; a step 302 of acquiring a theoretical model of the magnetic field of interest of one of the MF sources of the medical device; a step 303 of acquiring measurement entries of the magnetic field of interest (e.g., the magnetic field of a single one of the MF sources of the medical device, when the other MF sources are turned off); a step 304 (similar to step 202) of estimating errors between the measurement entries and the theoretical model; a step 305 (similar to step 203) of adjusting (interpolating, calibration, etc.) the computer model based on the estimated errors to generate a real-world 3D magnetic field computer model; a step 306 (similar to step 204) of verifying the accuracy of the interpolated computer model; a step 307 (similar to step 205) of refining the interpolated computer model; a step 308 of repeating at least some of the previous steps (e.g., steps 302 to 307) for at least some of the other MF sources (e.g., the other magnetic field heads) of the acquired medical device to generate a corresponding computer model (e.g., a real-world 3D magnetic field computer model for the magnetic field generated by each one of the magnetic heads); a step 309 of generating a complete/complex real-world computer model of a complete magnetic field comprising a combination of at least two magnetic fields (each produced by various MF sources); a step 310 of determining control parameters for each of the MF sources (or their controller) for producing the desired complete magnetic field based on the complete real-world computer model (e.g., to produce average magnetic field lines converging at an isocenter); and/or a step 311 of sending the control parameters to the medical device (e.g., to the interface of the medical device or to the controller(s) of its MF sources).

Step 309 can be done by considering the individual computer model (e.g., real-world 3D magnetic field computer model 222) of at least two MF sources 113 (e.g., magnetic heads) of the medical device 110. In some embodiments, these computer models can be combined together to generate a computer model of the full/complete magnetic field of the medical device that can represent/map the complex real-world magnetic field that can be generated. This can be achieved by considering the positions of each of the MF sources relative to the other MF sources or relative to an isocenter of the medical device.

In another example, when a medical device includes multiple magnetic field sources, the generating of a model of the magnetic field as described herein is repeated distinctly for each magnetic field source of the medical device, or can be repeated for a group of magnetic field sources (with one model of the magnetic field for the combined group of magnetic field sources).

In some embodiments, the individual computer models of the magnetic field generated by the various MF sources of the medical device may be used to adjust, calibrate and/or interpolate a theoretical complete computer model of the complete magnetic field produced with the medical device when a plurality or all of the individual magnetic fields are generated with the MF sources.

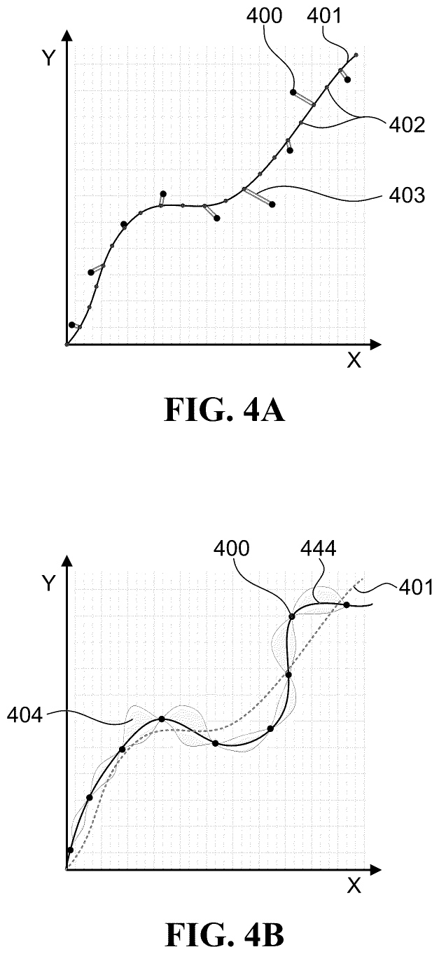

Reference is now made to FIGS. 4A and 4B that illustrate a possible embodiment of the correlation and interpolation that may be performed to generate, from a theoretical model, a real-world three-dimensional magnetic field model and/or to refine this computer model. It will be appreciated that the representations shown herein can be considered a simplified case of interpolation used to ensure clear understanding of the concept and method of interpolation and that it may not limit the anticipated variations, which may encompass all modifications, equivalents, combinations and alternatives falling within the spirit and scope of such interpolations.

FIG. 4A illustrates a possible embodiment of the adjusting of the theoretical model performed to obtain the real-world magnetic field model. FIG. 4A shows examples of the errors 403 between the real-world 400 values of the measurement entries and the default points 402 of the theoretical magnetic field line 401 of the initial/previous model.

In one embodiment, the adjusting can comprise minimizing the errors 403 by modifying (e.g., changing its shape, matching, interpolating) the theoretical magnetic field line 401 so that the default values 402 matches the real-world values 400.

FIG. 4B illustrates a possible embodiment of the adjusting of the computer model using the real-world measurements (e.g., real amplitude values) so as to adjust the previous computer model (e.g., theoretical model or real-world model to be refined) to be representative of the considered information/data/measurements. FIG. 4B shows examples of the coordinates of the correlated data/measurements 400, of the magnetic field line 401 of the initial/previous model, and the means of the normally distributed credible intervals 404 considered for the interpolation generate the adjusted/resulting/interpolated real-world magnetic field line 444 of the real-world computer model.

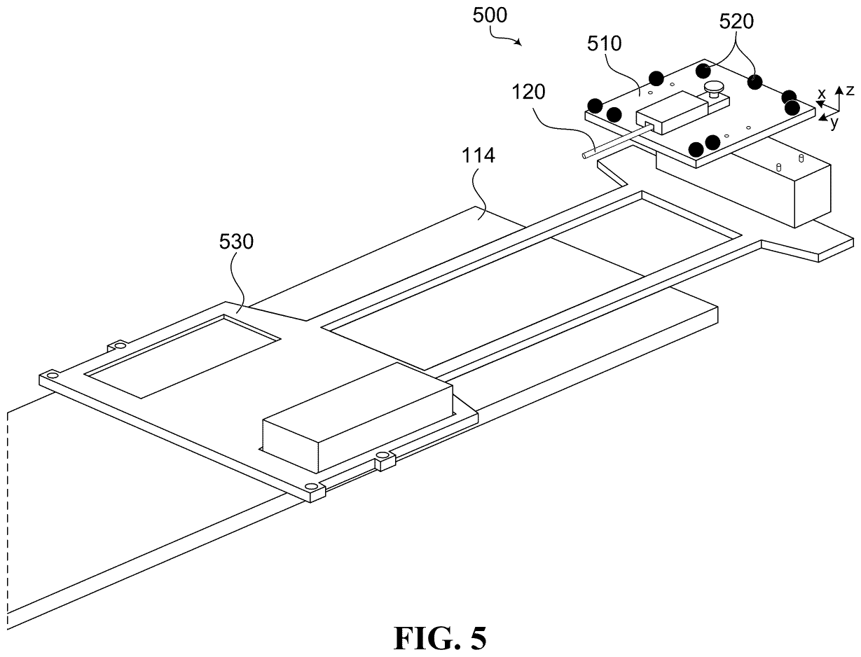

FIG. 5 shows an exemplary supporting device 500 for supporting and positioning a magnetic field measuring device 120 (e.g., a magnetometer) and the location markers 520. The supporting device 500 can comprise a supporting frame 510 (e.g., a position stage) to which location markers 520 (e.g., reflective sphere) can be coupled in an asymmetric layout. The tracking device 130 can track the 3D location of the location markers 520 in order to determine (e.g., by triangulation) the 3D location and optionally the orientation of the magnetic field measuring device 120. In some embodiments, the supporting device 500 can comprise a coupling frame 530 for coupling with the patient table 114 of the medical device 110 and for supporting of the position stage 510. It will be appreciated that the coupling frame 530 may be configured to be movable in height and/or along the length of the patient table 114 and/or along the width of the patient table, and can be rotated by 180 degrees (i.e., azimuthal rotation) to complete measurements at the other end of the patient table 114. The coupling frame 530 may include one or more apertures for receiving a complementary protrusion extending from the patient table 114 (e.g. in the example of FIG. 5, the coupling frame 530 is provided with two apertures for receiving a corresponding protrusion of the patient table 114). The coupling frame 530 may also include an extension portion located between and connecting, while separating, the portion of the coupling frame 530 with the one or more apertures, and the portion for supporting the position stage 510.

In some embodiments, the position of the supporting device 500 and of the magnetic field measuring device 120 can be moved (e.g., incrementally) within the working region by means of moving the patient table 114, which may be done with an actuator configured to control the position of the patient table 114.

### Exemplary Embodiment of the Magnetic-Field-Based Medical Device

A wide variety of medical devices can generate various magnetic fields. In fact, it will be appreciated that the proposed method and apparatus can be used and applied to any medical device that uses a magnetic field. While the following presents only a few possible apparatuses and embodiments of such medical devices, the presented exemplary embodiment may not limit the scope and possible applications of the proposed method and apparatus.

In one embodiment, the proposed method and apparatus can be used to generate a computer model of the real magnetic field produced by a least one of the magnetic generating components (e.g., magnetic heads) of a system for steering magnetotactic entities in a subject, namely for the purposes of harnessing the magnetotactic entities for therapy, imagery, diagnostics, etc. For instance, such magnetotactic entities may be self-propelling bacteria that follow a direction of a magnetic field (e.g. due to the presence of magnetosomes therein). The preferred medical device can enable the steering of these magnetotactic entities in a subject, once they have been introduced into the subject (e.g., by injection or otherwise).

The preferred medical device, which may be or be similar to the one described in the International application No. WO2023087093, incorporated herein by reference, can employ a magnetic field mainly for directional control of magnetotactic entities without inducing a displacement force to the entities. In such devices, the magnetic field may be used for directional control or steering of the magnetotactic entities. The system may be configured to provide sufficient space (e.g., more than 1.5 meters between the centers of each of the support arms for a human) for the subject laid out on an operating table surrounded by the magnetic sources of the system. The space provided for the patient table and the subject/patient can be referred to as a working region of the medical device and may be utilized by the operator to position the magnetic measuring device at various locations within this working region.

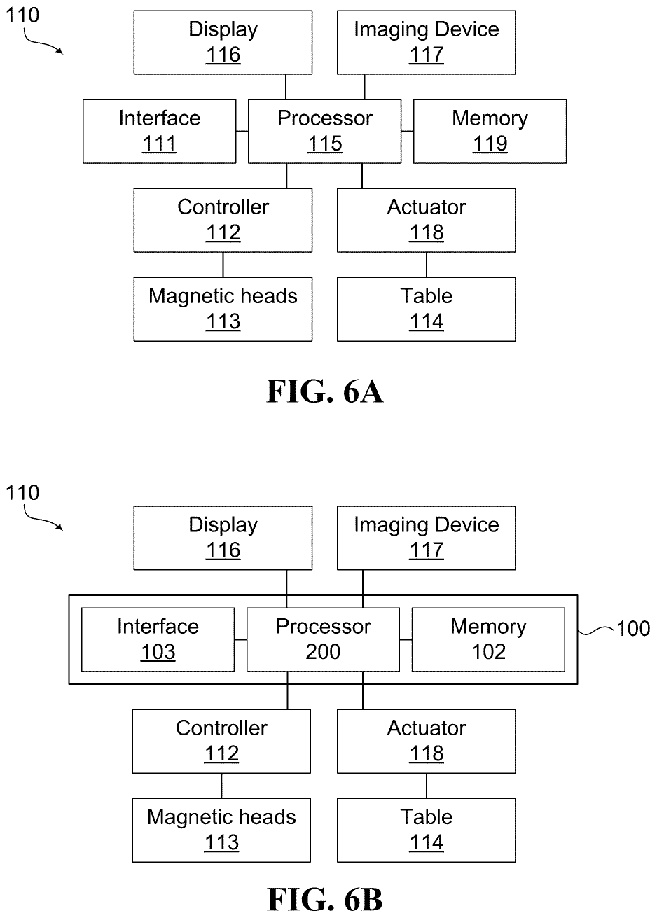

Reference is now made to FIG. 6A, illustrating an exemplary computer architecture of an exemplary system (medical device 110) for controlling magnetotactic entities, interacting with certain of the components of the system.

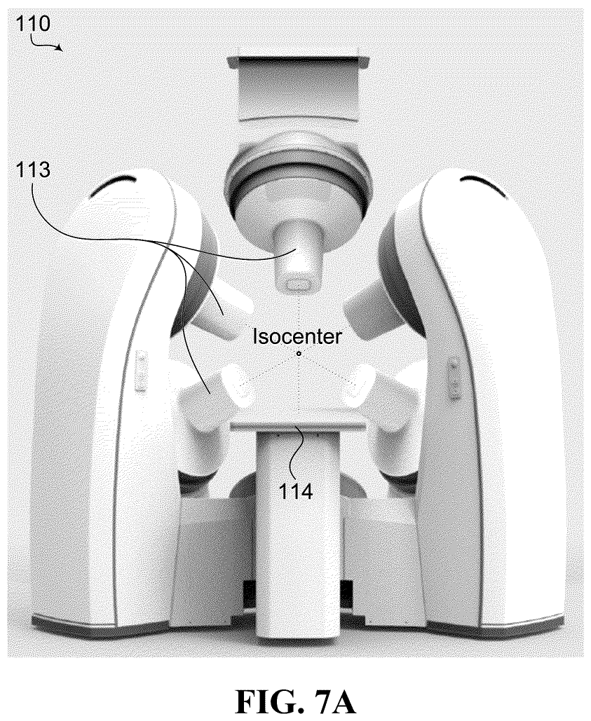

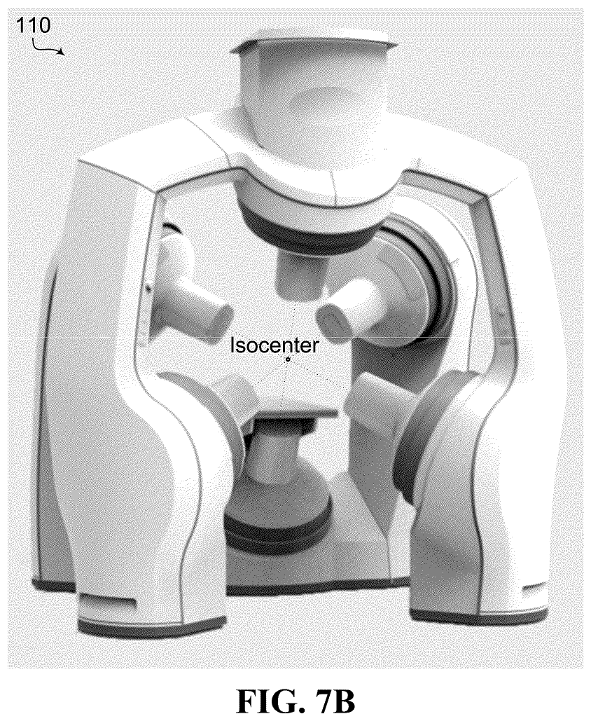

Such an architecture can be comprised in the exemplary medical device 110 illustrated in FIGS. 7A and 7B, which respectively present a drawing of a front view and a perspective view of all six magnetic field sources 113 (e.g. magnetic heads 113 having a coil contained within a housing, the coil generating a magnetic field when a current passes therethrough) and the patient table of an exemplary magnetic-field-based medical device for steering magnetotactic entities.

In examples where the medical device includes magnetic field sources oriented at a point, an isocenter is defined as this point.

The complete/full magnetic field of the medical device 110, which can result from the combination/interaction of the various individual magnetic fields produces by each of the MF source 113 (e.g., magnetic head), may be controlled/configured to ensure precise and effective guiding of magnetotactic entities (e.g. towards a convergence point or zone for the magnetotactic entities).

The system 110 includes a processor 115, memory 119 (e.g., on a printed circuit board—PCB) and a controller 112 (e.g., including a current amplifier).

The system 110 may include a user input interface 111 and/or a display 116. The computer may include an actuator 118.

The processor 115 may be a programmable processor. In this example, the processor 115 is shown as being unitary, but the processor may also be multicore, or distributed (e.g., a multi-processor).

The computer readable memory 119 may be used to store program instructions and data that can be used by the processor 115. The memory 119 may include non-transitory storage to store the program instructions. The computer readable memory 119, though shown as unitary for simplicity in the present example, may comprise multiple memory modules and/or caching. In particular, it may comprise several layers of memory such as a hard drive, external drive (e.g. SD card storage) or the like and a random-access memory (RAM) module. The RAM module may store data and/or program code currently being, recently being, or soon to be processed by the processor 115 as well as cache data and/or program code retrieved from non-transitory memory, e.g., a hard drive. A hard drive may store program code and be accessed to retrieve such code for execution by the processor 115 and may be accessed by the processor 115 to store magnetic head 113 sequences for generating a convergence point for the magnetotactic entities, and imaging data from the imaging device 117, as explained herein. The memory 119 may have a recycling architecture for storing, for instance, imaging data from the imaging device 117, coordinates for steering the magnetotactic entities, etc., where older data files can be deleted when the memory 119 is full or near being full, or after the older data files have been stored in memory 119 for a certain time.

The user input interface 111 can be in communication with the processor 115. The user input interface 111 may allow for a user to provide input to the system 110, such as controlling the magnetic heads 113, moving the table 114 and/or activating the imaging device 117. The user input interface 111 may be one or more of a touchscreen, one or more knobs, a keyboard, a mouse, a joystick, etc.

The processor 115, the memory 119 and the user input interface 111 may be linked via BUS connections.

The display 116 provides visual information to the user of the system 110, such as imaging data generated by the imaging device 117, values for the strength of the magnetic field generated by one or more pairs of magnetic heads 113, the location of the convergence point for steering the magnetotactic entities, overlaid, e.g., over an image of the subject generated by, for instance, the imaging device 117. The display 116 may also provide a graphical user interface for the user for controlling the system 110. The display 116 may also include a functionality of a user input interface 111, being configured as a touchscreen.

The one or more actuators 118 controls the position of the table 114. The one or more actuators 118, upon receiving commands from the processor 115, may cause the table to move up-or-down, side-to-side, forwards-or-backwards, or rotate. The one or more actuators 118 may be any combination of pneumatic, hydraulic, supercoiled, electric, rotary, linear, etc.

One or more controllers 112 (e.g., including current amplifiers) may be present for controlling the flow of electric current to the one or more magnetic heads 113, in order to cause the generation, or modifying the generation of the magnetic field generated by the one or more magnetic heads 113 when powered, as described in further detail below.

The user may control the system 110 by providing input via the user input interface 111. For instance, the user may provide input for activating one or more of the magnetic head pairs (or this may be done implicitly by the user designating a target in the subject). The processor 115 can receive the input for turning on one or more of the magnetic heads and can send commands to cause the controller 112 to open one or more switches and/or modulate current being directed to the one or more magnetic head pairs (e.g., through current amplifiers), as described in further detail below. In some embodiments, where the user provides a target for steering the magnetotactic entities, the processor 115 may retrieve from memory 119 one or more commands or functions to identify the appropriate magnetic head pairs 113 to turn on, the appropriate current to be provided to the one or more magnetic heads, and/or if the current is to remain constant, and/or if the magnetic field generated by each of the magnetic heads 113 is to remain constant or is to fluctuate in a time multiplexed manner, as explained in patent U.S. 9,905,347. In some embodiments, upon the user selecting a target in a subject for the steering of the magnetotactic entities, the processor 115 may also generate one or more commands transmitted to the actuator 118 in order to displace the table 114, and the user positioned thereon, for positioning the subject with respect to the convergence point that may be generated by the pairs of magnetic heads 113.

The user may also provide input via the user input interface 111 to displace the table 114. This input is received by the processor 115, where the processor 115 retrieves from memory 119 commands or functions to calculate table adjustment(s) and sends commands corresponding to the user input to the actuator 118 to cause the table 114 to be displaced.

The user input interface 111, processor 115 and memory 119 may be used to control the imaging device 117. However, in some embodiments, the imaging device 117 may have a separate computer, including a user input interface, for interacting with same.

Reference is now made to FIG. 6B, illustrating an exemplary computer architecture of an exemplary system (medical device 110) comprising an embodiment of the proposed computing device 100. In some embodiments, the computing device 100 can be integrated in any embodiment of the medical device 110. FIG. 6B shows an embodiment of the medical device similar to the one previously described and illustrated in FIG. 6A where the some of its components are the corresponding components/modules of the proposed computing device 100.

In some embodiments of the medical device 110, the user input interface 111 can be the interface 103 of the computing device 100 that can include/perform the functionalities of the user input interface 111 as described above.

In some embodiments the processor 115 of the medical device 110 can be the processor 200 of the computing device 100 that can include/perform the functionalities of the processor 115 of the medical device 110 as previously described.

In some embodiments the memory 119 of the medical device 110 can be the processor 102 of the computing device 100 that can include/perform the functionalities of the memory 119 of the medical device 110 as described above.

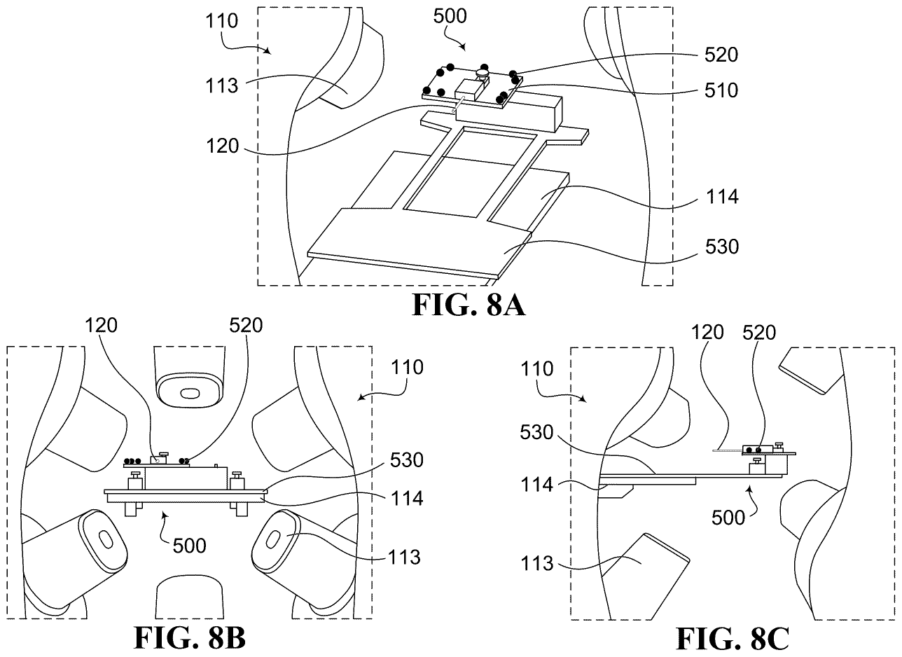

Reference is now made to FIGS. 8A, 8B and 8C that show schematic representations of the supporting device 500 that can be used to support and to position a magnetic field measuring device 120 and the location markers 520 for acquiring the measurement entries (e.g., at least comprising a magnetic field amplitude and positional coordinates of a corresponding measurement location) coupled to the top of the patient table 114 of an exemplary embodiment of the medical device 110 comprising various magnetic field sources (i.e., magnetic heads 113). These Figures respectively show a perspective view, a front view and a side view. In such a setup/configuration, or the like, the required acquisitions of the measurement entries can be done at various locations within the working region (e.g., the region/space between the various magnetic heads provided for the patient and the patient table) of the medical device.

Although the invention has been described with reference to preferred embodiments, it is to be understood that modifications may be resorted to as will be apparent to those skilled in the art. Such modifications and variations are to be considered within the purview and scope of the present invention.

Representative, non-limiting examples of the present invention were described above in detail with reference to the attached drawing. This detailed description is merely intended to teach a person of skill in the art further details for practicing preferred aspects of the present teachings and is not intended to limit the scope of the invention. Furthermore, each of the additional features and teachings disclosed above and below may be utilized separately or in conjunction with other features and teachings.

Moreover, combinations of features and steps disclosed in the above detailed description, as well as in the experimental examples, may not be necessary to practice the invention in the broadest sense, and are instead taught merely to particularly describe representative examples of the invention. Furthermore, various features of the above-described representative examples, as well as the various independent and dependent claims below, may be combined in ways that are not specifically and explicitly enumerated in order to provide additional useful embodiments of the present teachings.

What is claimed is

1. A computing device for representing, with a computer model, a magnetic field generated by a magnetic field source of a magnetic-field-based medical device, comprising:
an input interface for receiving a plurality of magnetic field measurement entries of said magnetic field;
a processor; and
memory storing program code that, when executed by said processor, causes said processor to:
receive a theoretical three-dimensional model of said magnetic field generated by said magnetic field source;
receive, via said input interface, said plurality of magnetic field measurement entries, wherein each magnetic field measurement entry of said plurality of magnetic field measurement entries comprises an amplitude value of said magnetic field and a corresponding positional coordinate;
estimate errors of amplitude between default amplitude values of said theoretical three-dimensional model and corresponding said amplitude values of said plurality of magnetic field measurement entries based on said positional coordinates of said plurality of magnetic field measurement entries; and
adjust default values of said theoretical three-dimensional model based on said estimated errors of amplitude, resulting in a real-world three-dimensional magnetic field model from said theoretical three-dimensional model with adjusted amplitude values, whereby said real-world three-dimensional magnetic field model comprises a constellation of adjusted amplitude values in a space representative of said magnetic field generated by said magnetic field source.

2. The computing device of claim 1, wherein said estimating comprises calculating a difference between said default values of said magnetic field of said theoretical model and said amplitude values of said magnetic field of said plurality of magnetic field measurement entries through an interpolation method.

3. The computing device of claim 2, wherein said adjusting is based on a Kriging interpolation method.

4. The computing device of any one of claims 1 to 3, further comprising said medical device.

5. The computing device of any one of claims 1 to 4, wherein said input data further comprises a value of orientation of said magnetic field, and wherein said estimating errors of amplitude further uses said value of orientation of each of said correlated said input data.

6. The computing device of any one of claims 1 to 5, wherein said program code, when executed by said processor, further causes said processor to verify an accuracy of said real-world three-dimensional magnetic field model of said magnetic field.

7. The computing device of claim 6, wherein said verifying an accuracy of said real-world three-dimensional magnetic field model of said magnetic field comprises:
receiving at least one additional magnetic field measurement entry comprising an amplitude value of said magnetic field and a corresponding positional coordinate;
estimating at least one additional error of amplitude between said default amplitude values of said real-world three-dimensional magnetic field model and at least one corresponding additional real-world amplitude value, wherein said at least one corresponding additional real-world amplitude value is determined from at least one additional magnetic field measurement entry including a corresponding positional coordinate; and
determining if said at least one additional error satisfies a verification threshold value to verify said accuracy of said real-world three-dimensional magnetic field model of said magnetic field.

8. The computing device of any one of claims 1 to 7, wherein said program code, when executed by said processor, further causes said processor to refine said real-world three-dimensional magnetic field model of said magnetic field.

9. The computing device of claim 6 or 7, wherein said program code, when executed by said processor, further causes said processor to refine said real-world three-dimensional magnetic field model of said magnetic field when said real-world three-dimensional magnetic field model of said magnetic field does not satisfy said verification of said accuracy of said real-world three-dimensional magnetic field model of said magnetic field.

10. The computing device of claim 8 or 9, wherein said refining of said real-world three-dimensional magnetic field model of said magnetic field comprises:
receiving said at least one additional magnetic field measurement entry comprising said amplitude value of said magnetic field and said corresponding positional coordinate;
estimating said at least one additional error of amplitude between said amplitude values of said real-world three-dimensional magnetic field model and said at least one corresponding real-world amplitude value of said at least one additional magnetic field measurement entry; and
adjusting said amplitude values of said real-world three-dimensional magnetic field model based on said at least one estimated additional error resulting in a refined real-world three-dimensional magnetic field model.

11. The computing device of claim 9, wherein said verifying is repeated after each of said refining to verify an accuracy of said resulting refined real-world three-dimensional magnetic field model and wherein said refining of said real-world three-dimensional magnetic field model of said magnetic field is repeated until said refined real-world three-dimensional magnetic field model satisfies said verification threshold.

12. The computing device of any one of claims 1 to 11, wherein said data is acquired with:
a magnetic field measuring device able to measure an amplitude of a magnetic field; and
a tracking device for identifying a location of said magnetic field measuring device;
wherein said magnetic field measuring device is used for determining said amplitude value of said magnetic field, and wherein said tracking device is used to measure said location of said magnetic field measuring device to determine said corresponding positional coordinates.

13. The computing device of claim 12, wherein said magnetic field measuring device further measures said value of orientation of said magnetic field.

14. The computing device of claim 12 or 13, wherein said magnetic field measuring device is a gaussmeter.

15. The computing device of any one of claims 12 to 14, wherein said magnetic field measuring device is coupled to at least one location marker that is trackable by said tracking device and wherein said tracking device determines a location of said at least one location marker to determine said location of said magnetic field measuring device.

16. The computing device of claim 15, wherein said at least one location marker is mounted on a frame coupled to said magnetic field measuring device, wherein said at least one location marker is positioned and said frame is shaped so as to reduce uncertainties of said location.

17. The computing device of any one of claims 12 to 16, wherein said magnetic field measuring device is coupled to said medical device for said measuring said amplitude value of said magnetic field.

18. The computing device of claim 17, wherein said a magnetic field measuring device is coupled to said medical device by way of a position stage, wherein said position stage is used to change said location of said magnetic field measuring device relative to said magnetic field of said magnetic field source of said medical device.

19. The computing device of claim 17 or 18, wherein said magnetic field measuring device is coupled to a patient table of said medical device and wherein said table is moved to change said location of said magnetic field measuring device relative to said magnetic field of said magnetic field source of said medical device.

20. The computing device of any one of claims 12 to 19, wherein said location of said magnetic field measuring device is comprised within a working region of said medical device.

21. The computing device of any one of claims 1 to 20, wherein said input interface is connectable to another computing device or another memory storing said data and providing said data to said processor,

22. The computing device of any one of claims 15 to 21, wherein said at least one location marker is a reflective marker, and wherein said tracking device is a stereo camera tracking said location of one or more of said at least one location marker to determine said location of said magnetic field measuring device.

23. The computing device of any one of claims 15 to 22, wherein said at least one location marker comprises at least three location markers, wherein said corresponding positional coordinates are determined by triangulation using said locations of said at least three location markers determined by said tracking device.

24. The computing device of any one of claims 1 to 23, wherein said magnetic field source is a first magnetic field source, wherein said representation of said magnetic field of said magnetic field source of said medical device is used with a representation of a magnetic field of at least one other magnetic field source of said medical device to generate a computer model of a combined magnetic field comprising said magnetic field of said first magnetic field source and of at least said magnetic field of said at least one other magnetic field source.

25. The computing device of claim 24, wherein each of said first magnetic field source and said at least one other magnetic field source comprises at least one electromagnet.

26. The computing device of claim 25, wherein said magnetic-field-based medical device comprises three pairs of magnetic field sources comprising said first magnetic field source and wherein said at least one other magnetic field source comprises five magnetic field sources.

27. The computing device of claim 26, wherein said three pairs of magnetic field sources is orthogonal with respect to each of said other two pairs of said three pairs.

28. The computing device of any one of claims 1 to 27, wherein said medical device is a system for steering self-propelled magnetotactic entities in a subject.

29. A method of representing a magnetic field of a magnetic source of a medical device with a computer model of said magnetic field, comprising:
acquiring, for a plurality of locations, an amplitude value of said magnetic field and corresponding positional coordinates of each of said locations;
acquiring a theoretical three-dimensional model of said magnetic field;
estimating errors of amplitude between said default values of said theoretical three-dimensional model and said corresponding amplitude values of said plurality of magnetic field measurement entries based on said positional coordinates of said plurality of magnetic field measurement entries; and
adjusting default values of said theoretical three-dimensional model based on said estimated errors, resulting in a real-world three-dimensional magnetic field model of said magnetic field of said magnetic source of said medical device from said theoretical three-dimensional model with adjusted amplitude values.

30. The method of claim 29, further comprising:
providing said medical device; and
generating a magnetic field with a magnetic field source of said medical device; and
wherein said acquiring said amplitude value of said magnetic field and said corresponding positional coordinates comprises:
providing a magnetic field measuring device able to measure an amplitude of said magnetic field;
providing a tracking device able to determine a location of said magnetic field measuring device;
measuring, for each of said plurality of locations, said amplitude value of said magnetic field using said magnetic field measuring device; and
acquiring, for each of said plurality of locations, said corresponding positional coordinates of each of said measured amplitude values by determining, using said tracking device, a location of said magnetic field measuring device when completing each of said measuring of said amplitude values.

31. The method of claim 29 or 30, further comprising at least one of:
verifying an accuracy of said real-world three-dimensional magnetic field model of said magnetic field; and
refining said real-world three-dimensional magnetic field model of said magnetic field.

32. The method of claim 31, wherein said refining is performed if said real-world three-dimensional magnetic field model does not satisfy said verifying said accuracy.

33. The method of claim 31 or 32, wherein said verifying comprises:
receiving at least one additional amplitude value of said magnetic field for at least one additional location and additional corresponding positional coordinates;
for each of said at least one additional amplitude value, estimating an additional error of amplitude between said at least one additional amplitude value and an amplitude of said real-world three-dimensional magnetic field model based on positional coordinates of said real-world three-dimensional magnetic field model; and
determining if each of said estimated additional error of amplitude satisfies a verification threshold value to verify an accuracy of said real-world three-dimensional magnetic field model.

34. The method of claim 31 or claim 32, wherein said refining comprises:
providing at least one additional amplitude value of said magnetic field for at least one additional location and additional corresponding positional coordinates;
for each on said at least one additional amplitude value, estimating an additional error of amplitude between said at least one additional amplitude value and an amplitude of said real-world three-dimensional magnetic field model based on positional coordinates of said real-world three-dimensional magnetic field model; and
adjusting said amplitude of said real-world three-dimensional magnetic field model based on said estimated additional error of amplitude.

35. The method of claim 34, wherein said refining is repeated, with at least one further additional amplitude value of said magnetic field for at least one further additional location and further additional corresponding positional coordinates, until said real-world three-dimensional magnetic field model satisfies said verifying said accuracy.

36. The method of any one of claims 29 to 35, further comprising generating a complete three-dimensional magnetic field model of a complete magnetic field of said medical device generated by a plurality of said magnetic field sources by combining said real-world three-dimensional magnetic field model of each of said plurality of said magnetic field sources.

37. The method of any one of claims 29 to 36, further comprising sending said control parameters to controllers of said medical device, each controlling one magnetic field source of said plurality of magnetic field sources.

38. A medical system comprising magnetic field sources of which said magnetic field of each of said magnetic field sources has been defined by performing said method as defined in any one of claims 29 to 37.
